(19) United States
(12) Patent Application Publication
(10) Pub. No.: US 2024/0415872 A1
(43) Pub. Date: Dec. 19, 2024

(54) OPHTHALMIC FORMULATIONS FOR TREATMENT OF PRESBYOPIA, DRY EYE DISEASE AND COMPUTER VISION SYNDROME

(71) Applicant: VSY BIYOTEKNOLOJI VE ILAC SANAYI ANONIM SIRKETI, Istanbul (TR)

(72) Inventors: Efe CAN, Mersin (TR); Faruk OYTUN, Istanbul (TR)

(73) Assignee: VSY BIYOTEKNOLOJI VE ILAC SANAYI ANONIM SIRKETI, Istanbul (TR)

(21) Appl. No.: 18/702,951

(22) PCT Filed: Dec. 31, 2021
(86) PCT No.: PCT/TR2021/051681

§ 371 Date: Apr. 19, 2024

(30) Foreign Application Priority Data
Oct. 19, 2021 (TR) 2021/016287

(51) Int. Cl. A61K 31/728; A61K 9/00; A61K 9/08; A61K 45/06; A61K 47/02; A61K 47/22; A61K 47/24; A61K 47/26

(52) U.S. Cl. CPC A61K 31/728; A61K 9/0048; A61K 9/08; A61K 45/06; A61K 47/02; A61K 47/22; A61K 47/24; A61K 47/26

(57) ABSTRACT

An ophthalmic formulation for the treatment of presbyopia, dry eye disease and computer vision syndrome, and a method for preparing the same is provided. The ophthalmic formulation to be applied topically is to treat symptoms caused by dry eye by rehabilitation and prevention of ocular surface damage, and to provide clear vision at close range by strengthening the contraction of the ciliary muscle and reducing the pupil size (pharmacological miosis), enhancing accommodation and increasing the depth of focus

### CROSS REFERENCE TO THE RELATED APPLICATIONS

This application is the national phase entry of International Application No. PCT/TR2021/051681, filed on Dec. 31, 2021, which is based upon and claims priority to Turkish Patent Application No. 2021/016287, filed on Oct. 19, 2021, the entire contents of which are incorporated herein by reference.

### TECHNICAL FIELD

The present invention relates to ophthalmic formulations for the treatment of presbyopia, dry eye disease and computer vision syndrome, and a method for preparing the same.

### BACKGROUND

Presbyopia is the loss of the lens' flexibility and ability to adapt due to advancing age resulting in visual impairment, especially at close range. Although there is no known apparent cause, it is thought to be the result of age-related deformation in the muscle fibers that are effective in adaptation ability of the lens of the eye. Presbyopia is the physiological inadequacy of accommodation associated with aging of the eye, resulting in a progressive deterioration of the eye's ability to focus clearly on close objects. Presbyopia begins in every person over the age of 45 on average.

Although it is similar to hyperopia in general, unlike the problems caused by structural defects of the eyeball in hyperopia, age-related hardening of the lens and inability to focus on nearby objects are experienced in presbyopia. Progression of the disease causes inability to see clearly for a long time or a feeling of pain in the forehead, temporal and around the eyes due to the adaptation effort of the eye. Individual's effort to see nearby objects may cause difficulties also in seeing distant objects clearly over time.

Reading glasses, multifocal intraocular or contact lenses and refractive surgery are used for presbyopia. Presbyopia can be partially eliminated with the use of glasses (reading glasses) or contact lenses, and it can be prevented or slowed down with various drugs and vitamin supplements. On the other hand, another approach is to apply a surgical treatment process. In the surgical method, procedures such as applying laser to the cornea or placing a lens into the eye are performed.

Dry eye is a multifactorial disease of the ocular surface characterized by loss of homeostasis of the tear film accompanied by ocular symptoms in which instability and hyperosmolarity of the tear film, ocular surface inflammation and damage, and neurosensory abnormalities play an etiological role. The prevalence of dry eye worldwide varies between 5.5% and 33% on average. This number is increasing day by day, especially due to the increase in human life expectancy, the use of contact lenses, the use of devices with screens and computers, the use of drugs and refractive surgery.

Computer vision syndrome, also called digital eye fatigue, describes a group of eye and vision-related problems which result from prolonged use of computers, tablets, e-readers, and mobile phones, and many people experience eye discomfort and vision problems when viewing digital screens for long periods of time.

Dry eye, computer vision syndrome and presbyopia cause burning, stinging, watering, irritation, foreign body feeling, fatigue, headache, blurred vision, nearsightedness, pain and light sensitivity in the eyes, and significantly affect the life quality. In the current method, artificial tears, which are symptomatically effective, are used for the treatment of dry eye.

European patent document no EP3517100, an application known in the state of the art, relates to formulations and methods of use for eye drop formulations comprising Carboxymethyl cellulose (CMC) and hyaluronic acid (HA) which have an improved distribution on the cornea.

United States patent document no US2017007637, an application known in the state of the art, relates to a pharmacological ophthalmic composition for use in the correction of presbyopia.

International patent document no WO2008071524, an application known in the state of the art, relates to physiological adjuvants or drugs in the form of eye drops which are useful in the treatment of corneal diseases.

European patent document no EP2758047, an application known in the state of the art, discloses compositions for the treatment of presbyopia, mild hyperopia and irregular astigmatism and production method thereof.

European patent document no EP3681500, an application known in the state of the art, discloses the use of pilcarbine hydrochloride for the treatment of various ocular conditions, including presbyopia.

International patent document no WO2010/135731, an application known in the state of the art, discloses compositions and methods for the treatment or reduction of presbyopia.

The United States patent document U.S. Ser. No. 10/993,932, an application known in the state of the art, discloses methods and compositions for the treatment of certain ocular disorders, including presbyopia.

### SUMMARY

The objective of the present invention is to provide clear vision at close range by strengthening the contraction of the ciliary muscle and reducing the pupil size (pharmacological miosis), enhancing accommodation and increasing the depth of focus, with an ophthalmic formulation to be applied topically, unlike the current method. It is aimed to treat the symptoms of dry eye by rehabilitation and prevention of ocular surface damage. Along with all these effects, it is aimed to eliminate the eye fatigue and headache caused by the difficulty in near vision and the effort to see near in computer vision syndrome, and also the symptoms caused by the dry eye.

### DETAILED DESCRIPTION OF THE EMBODIMENTS

The ophthalmic formulations according to the present invention are prepared in phosphate buffer or non-phosphate buffer solution, and comprise by weight
linear and/or crosslinked forms of sodium hyaluronate to be in range of 0.01%-5%, and
osmoprotectants in range of 0.001%-5%,
vitamin in range of 0.001%-5%,
antioxidant in range of 0.001%-5%,
mineral salt in range of 0.001%-5%,
phospholipids in range of 0.001%-5%,
electrolytes in range of 0.001%-5%,
drug delivery systems in range of 0.001%-5%,
cholinergics in range of 0.001%-5%,
acetylcholine esterase inhibitors in range of 0.001%-5%,
ocular decongestants in range of 0.001%-5%,
ocular non-steroid anti-inflammatory drugs in range of 0.001%-5%.

Within the scope of the invention, linear and/or crosslinked forms of sodium hyaluronate with a molecular weight of 1000 Da-7 MDa are used, and sodium hyaluronate with low and high molecular weight is important in terms of determining the viscosity, penetration and drug release properties of the final product. Linear-linked forms and crosslinked forms of sodium hyaluronate can be used both together and separately within the scope of the invention. Its linear form allows the drop to spread over the entire ocular surface, while its crosslinked form allows the drop to remain on the ocular surface for a longer time.

Components and derivatives which will create a synergistic effect can be used in the formulation.

Dry eye syndrome is accompanied by tear hyperosmolarity. Ocular epithelial cells get damaged due to increased osmolarity. Osmoprotectants are compounds which can penetrate through both sides of the double-layered lipid and provide osmotic balance and thereby protecting the cell from damage due to osmotic pressure. In one embodiment of the invention, the osmoprotectants in the formulation according to the invention are selected from the group comprising L-carnitine, betaine, putrescine, spermidine, spermine, glycinebetaine, b-alanine betaine, choline-O-sulfate, dimethyl-sulfonio propionate, trehalose, erythrole, fructan, mannitol, dextran, sorbitol, proline, ectoin and combinations thereof.

Antioxidants and vitamins protect the ocular surface cells against oxidative damage by means of neutralizing the reactive oxygen species (ROS) that occur during the inflammation which develops in dry eye and computer vision syndrome. Furthermore, several antioxidants (coenzyme Q10, curcumin, polyphenols) prolong the life cycle of cells by preventing apoptosis (programmed cell death). Again, many antioxidants improve the clinical findings of dry eye and computer vision syndrome by exhibiting anti-inflammatory effects. In one embodiment of the invention, the antioxidants in the formulation according to the invention are selected from a group comprising glutathione, allicin, astaxanthin, N-Acetylcarnosine (NAC), epigallocatechin gallate (EGCG), coenzyme Q10 (CoQ10), curcumin, polyphenols, quercetin, alpha lipoic acid, resveratrol, alpha tocopherol, pyruvate, carotene, beta carotene, trolox, hydroxytyrosol tyrosol, ferulic acid, caffeic acid, rutin, diosmin, melatonin, taurine, hypotaurine, and combinations thereof. In one embodiment of the invention, the vitamins in the formulation according to the invention are selected from the group comprising vitamin A derivatives such as retinal, retinol, pro-vitamin A, retinoic acid, vitamin B derivatives such as vitamin B1 (thiamine), vitamin B2 (riboflavin), vitamin B3 (nicotinamide), vitamin B5 (pantothenic acid), vitamin B6 (pyridoxine), vitamin B8 (biotin), vitamin B9 (folacin), vitamin B12 (cobalamins), vitamin C derivatives such as L-ascorbic acid, tetrahexyldecyl ascorbate, ascorbyl glucoside, ethylated ascorbic acid, ascorbyl palmitate, magnesium ascorbyl palmitate magnesium ascorbyl phosphate, calcium ascorbate, sodium ascorbate, and sodium ascorbyl phosphate, vitamin D and vitamin K derivatives, and combinations thereof.

In one embodiment of the invention, the mineral salts in the formulation according to the invention are selected from the group comprising zinc sulfate, zinc acetate, zinc glutamate, zinc PCA, calcium chloride, calcium carbonate and calcium phosphate, tricalcium citrate, calcium lactate, calcium lactate gluconate, calcium gluconate, magnesium oxide, magnesium citrate, magnesium gluconate, magnesium chloride, magnesium sulfate, magnesium lactate, magnesium aspartate hydrochloride, potassium chloride, potassium carbonate, selenium, lactate, citrate, borate, and combinations thereof.

Among the main objectives of the treatment of dry eye and computer vision syndrome is the treatment of tear deficiency. For this purpose, artificial tears are commonly used. An ideal artificial tear is typically a buffered solution and comprises the electrolytes found in natural tear. In an embodiment of the present invention, the electrolytes in the formulation according to the present invention are selected from a group comprising potassium, bicarbonate, sodium, chlorine, magnesium, manganese, calcium, and combinations thereof.

As it is known, the natural tear film comprises lipid layer in its outermost layer. The lipid layer plays a role in the stabilization of the liquid layer (prevents its loss due to evaporation). Phospholipids play an important role in tear stabilization. Furthermore, citicoline plays an active role in regeneration of cell membrane, which consists of a double-layered lipid, and has a regenerative effect. In one embodiment of the invention, the phospholipids in the formulation according to the present invention are selected from a group comprising phosphatidylcholine, phosphatidylethanolamine, phosphatidylserine, sphingomyelin, citicoline and combinations thereof.

The cornea has a strong hydrophobic structure. There are tight junctions between corneal epithelial cells called desmosomes and hemi-desmosomes. This structure of the cornea is the most important barrier for adequate penetration of aqueous solutions into the anterior chamber. Detergents which are generally quaternary ammonium derivatives (Benzalkonium chloride) are used so that the drugs penetrate into the eye at effective concentrations. However these components cause significant ocular surface damage. It is not suitable for patients currently with dry eye and computer vision syndrome to use topical drops with these preservatives in terms of the course of the disease. Drug delivery systems are needed to achieve therapeutic concentrations in the ciliary body and the iris without reaching toxic concentrations at the ocular surface. In one embodiment of the invention, the drug delivery systems in the formulation according to the present invention are selected from a group comprising liposomes, niosomes, nanoparticles, dendrimers, micelles, polymer-drug conjugates and combinations thereof.

Parasympathomimetic drugs activate the parasympathetic nervous system. When applied topically to the eye, they cause miosis (shrinking of the pupil) by causing the sphincter pupillary muscle in the iris to contract. They also reduce the tension in the zonules by strengthening the contraction of the ciliary body. In this way, as the tension on the crystalline lens decreases, its thickness and refractive ability increase. Synergy of these two effects facilitates near vision. Parasympathomimetic drugs are generally divided into two main classes: Cholinergics and acetylcholine esterase inhibitors. Cholinergics act by mimicking the effect of natural acetylcholine. On the other hand, acetyl choline esterase inhibitors act by increasing the amount of acetylcholine in the synapse by inhibiting acetyl choline esterase, which degrades acetylcholine at the neurosynaptic junction and makes it ineffective. In one embodiment of the invention, the cholinergics in the formulation according to the present invention are selected from a group comprising acetylcholine, carbachol, pilocarpine and combinations thereof. In one embodiment of the invention, the cholinesterase inhibitors in the formulation according to the present invention are selected from a group comprising rivastigmine, physostigmine, demecarium, neostigmine, pridostigmine and combinations thereof.

Non-steroidal anti-inflammatory drugs (NSAIDs) inhibit the cyclo-oxygenase enzyme and prevent the formation of arachidonic acid metabolites (prostaglandins and thromboxanes) that have pro-inflammatory effects. They exhibit anti-inflammatory effect by preventing redness, swelling, temperature increase and pain, which are the main markers of inflammation. When applied topically to the ocular surface, they treat the inflammation associated with dry eye and also reduce the burning, stinging and foreign body feeling that are among the symptoms of dry eye. Again, they reduce the pain and redness which can be caused especially by parasympathomimetic drugs. In one embodiment of the invention, the ocular non-steroidal anti-inflammatory drugs in the formulation according to the present invention are selected from a group comprising diclofenac, bromfenac, napefenac, flurbiprofen and combinations thereof.

Decongestants treat redness and edema by causing contraction of small and medium-sized vessels in the area they are applied. When applied topically to the ocular surface, they relieve redness caused by dry eye, inflammation, drug side effects, and allergies. In one embodiment of the invention, the ocular decongestants in the formulation according to the present invention are selected from a group comprising Naphazoline hydrofluoride, Tetrahydrozoline hydrofluoride and combinations thereof.

In one embodiment of the invention, the formulation according to the present invention is used for the treatment and prevention of presbyopia, dry eye disease and computer vision syndrome. In a preferred embodiment of the invention, the formulation according to the present invention can be used by dropping one drop every 4 hours.

In one embodiment of the invention, the formulation according to the present invention can be used in the form of disposable topical vials, reusable topical preservative-free bottles and tubes for gel form.

Unlike the current method, with an ophthalmic formulation to be applied topically, dry eye associated symptoms are treated by rehabilitation and prevention of ocular surface damage, and furthermore clear vision at close range is provided by strengthening the contraction of the ciliary muscle and reducing the pupil size (pharmacological miosis), enhancing accommodation and increasing the depth of focus.

The production method of the formulation according to the present invention comprises the steps of
Preparing buffer solution,
Filtering the buffer solution through a 0.2 micron filter,
Adjusting the pH of the buffer solution to the value range of 6.8-7.6,
Mixing the components to be used in the formulation in the buffer solution with the help of a mixer at 40-250 rpm for 1-10 hours,
Degassing the mixture under a vacuum,
Filtering the mixture through a 0.2 micron filter,
Filling the mixture into vials and bottles under sterile conditions,
Labelling and packing the final products.

What is claimed is:

1. An ophthalmic formulation, wherein the ophthalmic formulation is prepared in phosphate buffer or non-phosphate buffer solution, comprising:
sodium hyaluronate between 0.01% and 5% by weight, wherein the sodium hyaluronate is in linear form and/or crosslinked form,
an osmoprotectant between 0.001% and 5% by weight,
a vitamin between 0.001% and 5% by weight,
an antioxidant between 0.001% and 5% by weight,
a mineral salt between 0.001% and 5% by weight,
a phospholipid between 0.001% and 5% by weight,
an electrolyte between 0.001% and 5% by weight,
a drug delivery system between 0.001% and 5% by weight,
a cholinergic between 0.001% and 5% by weight,
an acetylcholine esterase inhibitor between 0.001% and 5% by weight,
an ocular decongestant between 0.001% and 5% by weight, and
an ocular non-steroid anti-inflammatory drug between 0.001% and 5% by weight.

2. The ophthalmic formulation according to claim 1, wherein the osmoprotectant is one or more selected from the group consisting of L-carnitine, betaine, putrescine, spermidine, spermine, glycinebetaine, b-alanine betaine, choline-O-sulfate, dimethyl-sulfonio propionate, trehalose, erythrole, fructan, mannitol, dextran, sorbitol, proline, and ectoin.

3. The ophthalmic formulation according to claim 1, wherein the antioxidant is one or more selected from the group consisting of glutathione, allicin, astaxanthin, N-Acetylcarnosine (NAC), epigallocatechin gallate (EGCG), coenzyme Q10 (CoQ10), curcumin, polyphenols, quercetin, alpha lipoic acid, resveratrol, alpha tocopherol, pyruvate, carotene, beta carotene, trolox, hydroxytyrosol tyrosol, ferulic acid, caffeic acid, rutin, diosmin, melatonin, taurine, and hypotaurine.

4. The ophthalmic formulation according to claim 1, wherein the vitamin is one or more selected from the group consisting of retinal, retinol, pro-vitamin A, retinoic acid, vitamin B1 (thiamine), vitamin B2 (riboflavin), vitamin B3 (nicotinamide), vitamin B5 (pantothenic acid), vitamin B6 (pyridoxine), vitamin B8 (biotin), vitamin B9 (folacin), vitamin B12 (cobalamins), L-ascorbic acid, tetrahexyldecyl ascorbate, ascorbyl glucoside, ethylated ascorbic acid, ascorbyl palmitate, magnesium ascorbyl palmitate magnesium ascorbyl phosphate, calcium ascorbate, sodium ascorbate, sodium ascorbyl phosphate, vitamin D, and vitamin K.

5. The ophthalmic formulation according to claim 1, wherein the mineral salt is one or more selected from the group consisting of zinc sulfate, zinc acetate, zinc glutamate, zinc PCA, calcium chloride, calcium carbonate and calcium phosphate, tricalcium citrate, calcium lactate, calcium lactate gluconate, calcium gluconate, magnesium oxide, magnesium citrate, magnesium gluconate, magnesium chloride, magnesium sulfate, magnesium lactate, magnesium aspartate hydrochloride, potassium chloride, potassium carbonate, selenium, lactate, citrate, and borate.

6. The ophthalmic formulation according to claim 1, wherein the electrolyte is one or more selected from the group consisting of potassium, bicarbonate, sodium, chlorine, magnesium, manganese, and calcium.

7. The ophthalmic formulation according to claim 1, wherein the phospholipid is one or more selected from the group consisting of phosphatidylcholine, phosphatidylethanolamine, phosphatidylserine, sphingomyelin, and citicoline.

8. The ophthalmic formulation according to claim 1, wherein the drug delivery system is one or more selected from the group consisting of liposomes, niosomes, nanoparticle, dendrimer, micelles, and polymer-drug conjugates.

9. The ophthalmic formulation according to claim 1, wherein the cholinergic is one or more selected from the group consisting of acetylcholine, carbachol, and pilocarpine.

10. The ophthalmic formulation according to claim 1, wherein the cholinesterase inhibitor is one or more selected from the group consisting gf rivastigmine, physostigmine, demecarium, neostigmine, and pridostigmine.

11. The ophthalmic formulation according to claim 1, wherein the ocular non-steroidal anti-inflammatory drug is one or more selected from the group consisting of diclofenac, bromfenac, napefenac, and flurbiprofen.

12. The ophthalmic formulation according to claim 1, wherein the ocular decongestant is one or more selected from the group consisting of Naphazoline hydrofluoride and Tetrahydrozoline hydrofluoride.

13. A method for treatment and prevention of presbyopia, dry eye disease or computer vision syndrome comprising the step of administering the ophthalmic formulation according to claim 1 to a subject in need.

14. A disposable topical vial, reusable topical preservative-free vial or tube for gel form comprising the ophthalmic formulation according to claim 1.

15. A method of producing the ophthalmic formulation according to claim 1, comprising the steps of
preparing buffer solution,
filtering the buffer solution through a 0.2 micron filter,
adjusting the pH of the buffer solution to a pH value between 6.8 and 7.6,
mixing the components to be used in the formulation in the buffer solution with a mixer at 40-250 rpm for 1-10 hours,
degassing the mixture under a vacuum,
filtering the mixture through a 0.2 micron filter,
filling the mixture into vials and bottles under sterile conditions,
labelling and packing the final products.
